(19) United States
(12) Patent Application Publication
(10) Pub. No.: US 2026/0249008 A1
(43) Pub. Date: Aug. 27, 2026

(54) Fluid Transfer Device and Method of Use Thereof for Caustic Medication Delivery

(71) Applicant: Becton, Dickinson and Company, Franklin Lakes, NJ (US)

(72) Inventors: Dorien Gemma Butter, Millcreek, UT (US); Jonathan Karl Burkholz, Salt Lake City, UT (US); Benjamin P. Hopwood-Cather, West Valley City, UT (US); Manish Kumar, Bengaluru (IN); Alison Nicole Anderson, Salt Lake City, UT (US); Ajay Suryavanshi, Pune (IN); Michael Glen Beeman, Salt Lake City, UT (US)

(21) Appl. No.: 19/062,457

(22) Filed: Feb. 25, 2025

(51) Int. Cl. A61M 5/19; A61M 5/178; A61M 25/00

(52) U.S. Cl. CPC A61M 5/19; A61M 25/0043; A61M 2005/1787; A61M 2025/0004; A61M 2025/0057; A61M 2205/33; A61M 2205/3324

(57) ABSTRACT

Provided herein is a system for intravenous delivery of a caustic medication to a patient. The system includes a catheter assembly, a fluid source containing a caustic medication, and a fluid transfer device coupleable to the fluid source and to the catheter assembly. The fluid transfer device includes a flexible tube, a housing configured to movably receive the flexible tube, and an advancement member configured to move relative to the housing to move the flexible tube between a first position and a second position, with the distal end of the flexible tube disposed past a distal tip of the catheter and able to transfer fluid from the fluid source to the patient when in the second position. The system is operable to control an intravenous location or dispersion of the caustic medication infused to the patient and/or a dilution of the caustic medication infused to the patient.

### BACKGROUND OF THE INVENTION

### Field of the Invention

The present disclosure relates generally to fluid transfer devices for use with intravenous (IV) catheters and, more specifically, to fluid transfer devices that are configured to enable administering of caustic medications through a peripheral intravenous catheter.

### Description of Related Art

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC) or peripherally inserted intravenous catheter (PICC), as examples.

Recent developments in the PIVC field have led to the emergence of technologies designed to facilitate fluid transfer (e.g., blood draw and fluid infusion) using an in-dwelling PIVC and accompanying fluid transfer device. The main method by which these fluid transfer devices work is by employing an introducer for inserting a flexible tube, probe, or other instrument through the catheter lumen of the PIVC, with the introducer being attached to a catheter adapter of the PIVC that provides for insertion of the flexible tube into the PIVC. For example, the catheter adapter may include a needleless access connector thereon by which the flexible tube may be introduced to provide access to the PIVC and into the patient's vasculature. In operation of the fluid transfer device, an advancement member is actuated by an operator relative to a housing to effectuate movement of the flexible tube within the housing and advance the flexible tube out therefrom for advancement into the in-dwelling PIVC. That is, the advancement member may be moved distally by the operator to cause a corresponding movement of the flexible tube relative to the housing, such that the flexible tube may be advanced out from the housing and into the in-dwelling PIVC.

With regard to the use of a VAD for purposes of fluid infusion, it is recognized that the infusion of certain caustic medications, such as chemotherapy drugs or certain antibiotics, has typically been performed via PICCs and CVCs, rather than PIVCs. That is, it is known that infusion of such caustic medications may damage the tunica intima layer of a vein, especially when delivery of such medications is to smaller diameter peripheral veins, such that caustic medications are typically infused to larger diameter veins/arteries via the use of PICCs and CVCs. While such fluid infusion is effective, it is recognized that it would be desirable to enable the infusion of caustic medications into a PIVC via a fluid transfer device as described above, as such fluid infusions could then be performed using an indwelling PIVC without the need for additional needle sticks.

Accordingly, a need exists in the art for a fluid transfer device that provides for infusion of caustic medications into an indwelling PIVC, while reducing the harmful effects of caustic medications on vein walls when delivered into peripheral veins. The fluid transfer device should enable variation in the location of intravenous administration of the caustic medication, with such distributed delivery lessening the risk of tissue damage to the vein. The fluid transfer device should also enable dilution of the caustic medication upon infusion, to again lessen the risk of tissue damage to the vein at the point of infusion. The fluid transfer device should still further allow for monitoring of a concentration of the caustic medication at different positions along the length of the flexible tube or near the tip of the PIVC, to allow clinicians to closely monitor and adjust positioning or dosage of the caustic medication.

### SUMMARY OF THE INVENTION

Provided herein is a system for intravenous delivery of a caustic medication to a patient. The system includes a catheter assembly including a catheter adapter and a catheter positionable intravenously in the patient. The system also includes a fluid source containing a caustic medication in fluid form and a fluid transfer device coupleable to the fluid source and to the catheter assembly. The fluid transfer device further includes a flexible tube configured to be inserted within the catheter and in fluid communication with the fluid source, a housing having a proximal end and a distal end and defining an inner volume configured to movably receive the flexible tube, a coupling device positioned at the distal end of the housing and configured to couple the housing to the catheter assembly, and an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer fluid from the fluid source to the patient when in the second position. The fluid transfer device is configured to control one or more of an intravenous location or dispersion of the caustic medication infused to the patient, and a dilution of the caustic medication infused to the patient.

In some embodiments, the flexible tube comprises a cylindrical outer wall, with the cylindrical outer wall having an outer diameter sized to be less than a minimum section of the catheter inner diameter, with sufficient wall thickness and stiffness to allow passage through the catheter.

In some embodiments, a gauge of the catheter is between 14 G and 26 G and a gauge of the flexible tube is less than the gauge of the catheter, such that a gap is provided between an outer surface of the flexible tube and an inner surface of the catheter.

In some embodiments, a width of the gap is between 0.0 inches and half of a catheter tip inner diameter.

In some embodiments, the catheter adapter comprises a proximal port and a side port, with the fluid transfer device connected to one of the proximal port or a side port via the coupling device.

In some embodiments, the system further includes a second fluid source coupled to the other of the proximal port or the side port and in fluid communication with the catheter, the second fluid source containing a non-caustic medication or saline solution.

In some embodiments, the non-caustic medication or saline solution is infused through the catheter to the patient via a sheath flow through the gap between the outer surface of the flexible tube and the inner surface of the catheter.

In some embodiments, the flexible tube comprises a plurality of diffuser holes formed therein at the distal end of the flexible tube.

In some embodiments, the plurality of diffuser holes is distributed along a length and/or circumference of the flexible tubing.

In some embodiments, the system further includes a sensor positioned at the distal end of the flexible tube or the distal tip of the catheter configured to measure a concentration of the caustic medication infused to the patient.

In some embodiments, the sensor comprises a pH sensor.

In some embodiments, the sensor comprises an osmolarity sensor.

In some embodiments, the fluid transfer device comprises a secondary catheter extending proximally out from the housing, the secondary catheter including a connector at a proximal end thereof, with the fluid source coupled to the connector on the secondary catheter.

Also provided herein is a method of intravenously delivering a caustic medication to a patient. The method includes connecting a fluid transfer device to a catheter assembly including a catheter adapter and a catheter having a distal tip that is indwelling intravenously in the patient, with the fluid transfer device comprising a flexible tube configured to be inserted within the catheter and in fluid communication with the fluid source, a housing having a proximal end and a distal end and defining an inner volume configured to movably receive the flexible tube, a coupling device positioned at the distal end of the housing and configured to couple the housing to the catheter assembly, and an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer fluid from the fluid source to the patient when in the second position. The method also includes connecting a fluid source containing a caustic medication in fluid form to the fluid transfer device such that the fluid source is fluidly connected with the flexible tube, actuating the advancement member to move the flexible tube from the first position to the second position and, with the flexible tube in the second position, operating the fluid source to administer the caustic medication to the patient, via the flexible tube. In administering the caustic medication to the patient, the fluid transfer device controls one or more of an intravenous location or dispersion of the caustic medication administered to the patient, and a dilution of the caustic medication administered to the patient.

In some embodiments, the method further includes connecting another fluid source to the catheter assembly via a side port of the catheter adapter and such that the another fluid source is fluidly connected with the catheter, wherein the another fluid source contains a non-caustic medication or saline solution, and operating the another fluid source to administer the non-caustic medication or saline solution to the patient, via the catheter.

In some embodiments, the non-caustic medication or saline solution is administered to the patient simultaneously with administration of the caustic medication, with the non-caustic medication or saline administered via a sheath flow through a gap between an outer wall of the flexible tube and an inner surface of the catheter.

In some embodiments, a size of the gap is between 0.0 inches and half of a catheter tip inner diameter.

In some embodiments, in administering the caustic medication to the patient, the caustic medication is infused through diffuser holes formed in an outer wall of the flexible tube, at the distal end of the flexible tube.

In some embodiments, the method further includes monitoring a parameter of the caustic medication during administering thereof via a sensor positioned at the distal end of the flexible tube or at the distal tip of the catheter, wherein the parameter comprises one of a pH or osmolarity of the caustic medication.

In some embodiments, the method further includes actuating the advancement member during administering of the caustic medication to move the distal end of the flexible tube to a plurality of positions that are all distal to the distal tip of the catheter, to vary an intravenous location within the patient for de-localized delivery of the caustic medication.

### BRIEF DESCRIPTION OF THE DRAWINGS

FIG. 1 is a perspective view of a catheter system, according to an aspect or embodiment of the present application;

FIG. 2 is a perspective view of a fluid transfer device of the system of FIG. 1, according to an aspect or embodiment of the present application;

FIG. 3 is an exploded view of the fluid transfer device of FIG. 2;

FIG. 4 is a side cross-sectional view of the fluid transfer device of FIG. 2, showing a flexible tube thereof in a first, retracted position;

FIG. 5 is a side cross-sectional view of the fluid transfer device of FIG. 2, showing the flexible tube in a second, extended position;

FIG. 6 is a cross-sectional view illustrating positioning of a flexible tube of the fluid transfer device of FIG. 2 and an IV catheter positioned within a vein;

FIG. 7 is a cross-sectional view illustrating positioning of a flexible tube of the fluid transfer device of FIG. 2 within an IV catheter;

FIG. 8 is a partial perspective view of the system of FIG. 1 showing the flexible tube of the fluid transfer device having a plurality of diffuser holes therein, according to an aspect or embodiment of the present application;

FIG. 9 is a partial perspective view of the system of FIG. 1 showing the flexible tube of the fluid transfer device having a plurality of diffuser holes therein, according to another aspect or embodiment of the present application;

FIG. 10 is a partial perspective view of the system of FIG. 1 showing the flexible tube of the fluid transfer device having a plurality of diffuser holes therein, according to another aspect or embodiment of the present application;

FIG. 11 is a partial perspective view of the system of FIG. 1 showing the flexible tube of the fluid transfer device having a sensor provided thereon, according to an aspect or embodiment of the present application;

FIG. 12 is a partial perspective view of the system of FIG. 1 showing the catheter of the catheter assembly having a sensor provided thereon, according to another aspect or embodiment of the present application; and

FIG. 13 is a schematic illustration showing positioning of distal end of a flexible tube of the fluid transfer device of FIG. 2 at a number of different intravenous locations, according to an aspect or embodiment of the present application.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

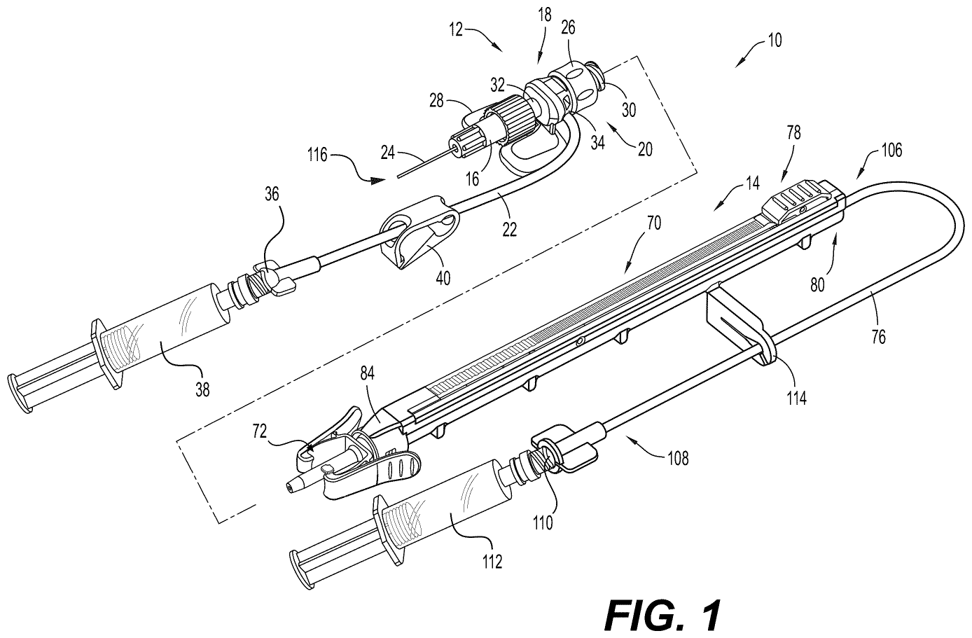

Referring to FIG. 1, shown is a non-limiting embodiment of a catheter system 10 for facilitating infusion of a caustic medication (e.g., chemotherapy drugs or certain antibiotics, as non-limiting examples) to a patient, according to one aspect or embodiment with which embodiments of the invention may be implemented. The catheter system 10 includes a catheter assembly 12 with which an associated fluid transfer device 14 may be used.

In accordance with one embodiment, the catheter assembly 12 includes a catheter hub 16 and a non-integrated extension set 18 that includes a catheter stabilizing catheter connector 20 (hereafter “catheter connector 20”), along with extension tubing 22. The catheter hub 16 receives a catheter 24 and is coupled to the catheter connector 20. In one aspect or embodiment, the catheter hub 16 may be an AccuCath™ catheter system commercially available from Becton, Dickinson and Company.

The catheter connector 20 is configured to be placed in contact with the skin surface of a patient at or near an insertion site of catheter 24. The catheter connector 20 can be any suitable shape, size, and/or configuration. In the illustrated non-limiting embodiment, the catheter connector 20 has a connector portion 26 and a stabilization portion 28. The connector portion 26 has a proximal port with a proximal coupler 30, a distal port with a distal coupler 32, and defines at least one lumen (not shown) extending through or otherwise in fluid communication with the couplers 30, 32. The proximal coupler 30 and/or the distal coupler 32 can be, for example, male or female luer locks and/or any other suitable coupler. In the illustrated embodiment, the proximal coupler 30 is provided as a needleless access connector (i.e., needle-free connector (NFC))—hereafter “needleless access connector 30”—that can be physically and fluidically coupled to peripheral devices such as fluid transfer device 14, which may comprise a PIVO™ device from Becton, Dickinson and Company, as a non-limiting example. The proximal coupler 30 presents a female luer connection/lock to which fluid transfer device 14 may be connected. The distal coupler 32 can be physically and fluidically coupled to catheter hub 16 such that the lumen of the connector portion 26 is at least selectively in fluid communication with the catheter 24. In some embodiments, an inner surface of the connector portion 26 can be configured to provide alignment, guidance, centering, etc., to an object or device (e.g., a blood draw catheter or the like, as described above) being advanced therethrough.

The connector portion 26 may also include and/or define one or more additional ports, such as a side port 34. Side port 34 can be included in and/or can be a part of the connector portion 26, the proximal coupler 30, the distal coupler 32, and/or a combination thereof. In some instances, positioning side port 34 in a desired position along a length of the connector portion 26, between the couplers 30, 32 can allow for a reduced length of the connector portion 26 and/or can facilitate flushing and/or fluid transfer via side port 34.

Side port 34 defines a lumen (not shown) that is in fluid communication with the lumen between distal coupler 32 and proximal coupler 30. In other words, the connector portion 26 and/or the side port 34 can include and/or define a first lumen (e.g., between distal coupler 32 and proximal coupler 30) and a second lumen (e.g., lumen defined by side port 34 and extension tubing 22). As such, side port 34 can provide access to the lumen between distal coupler 32 and proximal coupler 30, which in turn can provide access to a device (e.g., a VAD) that is coupled to the distal coupler 32 and/or can provide access to a portion of the body in which the VAD is at least partially disposed.

In some embodiments, the arrangement of side port 34 can be such that the connector portion 26 forms, for example, a Y-connector or a T-connector. More particularly, side port 34 can be disposed substantially perpendicular (e.g., about 90 degrees) to the lumen of the connector portion 26 and near or adjacent the proximal coupler 30. In non-limiting embodiments, side port 34 extends from connector portion 26 at an angle, relative to the lumen between distal coupler 32 and proximal coupler 30, that is not 90 degrees (e.g., side port 34 extends at an angle of, for example and without limitation, 15-165 degrees, with all values and subranges therebetween inclusive). In non-limiting embodiments, more than one side port 34 is included in connector portion 26.

As shown in FIG. 1, the side port 34 of the catheter connector 20 may be coupled to a distal end of extension tubing 22, such that the extension tubing 22 is in fluid communication with the lumen of the side port 34. A proximal end portion of the extension tubing 22 may comprise a proximal access port 36, which may be configured as a luer connection. In some embodiments, proximal access port 36 may include a needleless access connector provided thereon. According to aspects of the disclosure, the side port 34 and the extension tubing 22 can be and/or can form at least a portion of a fluid line that can be used to deliver fluid to the catheter assembly 12. For example, a (second) fluid source 38, such as a pre-filled syringe, may be fluidly connected to side port 34 and extension tubing 22 via proximal access port 36, to deliver fluid intravenously to the patient via catheter 24. In some embodiments, and as described in further detail below, the fluid source 38 may deliver a non-caustic medication or saline solution to catheter assembly 12. In some embodiments, a clamp 40 may be provided on the extension tubing 22, with the clamp 40 configured to selectively restrict flow through the extension tubing 22.

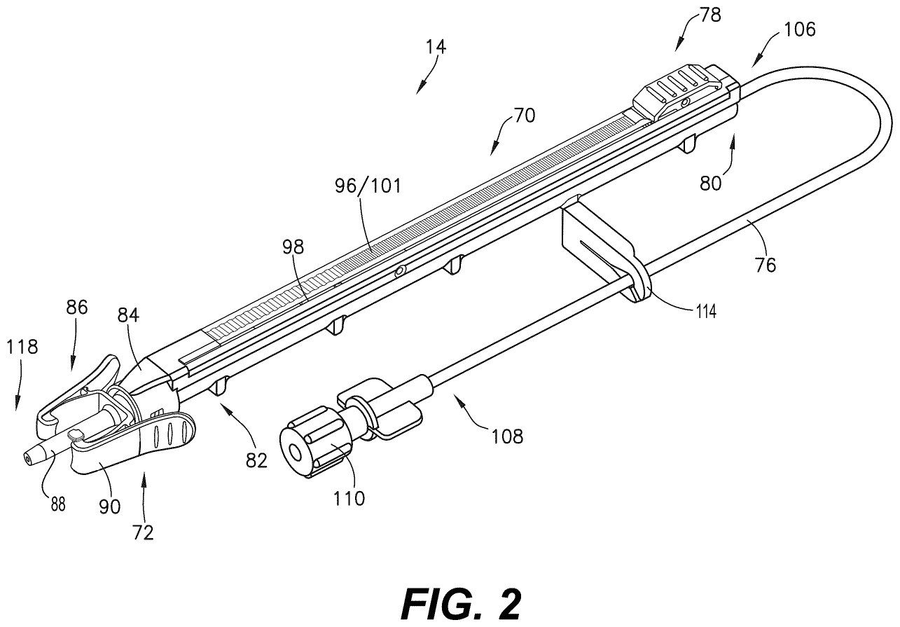

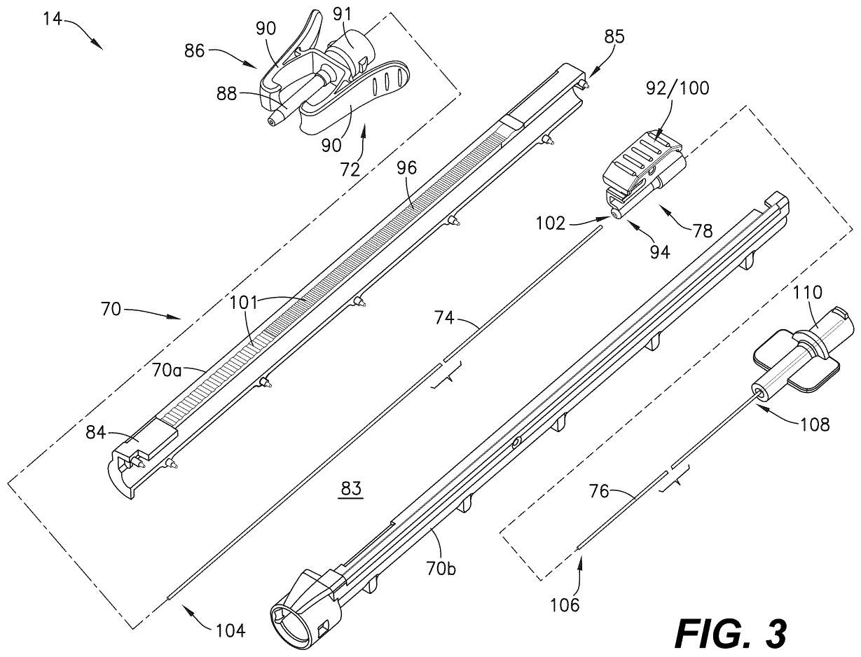

Referring still to FIG. 1 and now also to FIGS. 2 and 3, the fluid transfer device 14 (hereafter “fluid transfer device 14”) includes a housing 70, a coupling device 72, a flexible tube 74, a secondary catheter 76, and an advancement member 78. As described in further detail herein, a user can transition the fluid transfer device 14 from a first configuration to a second configuration to advance the flexible tube 74 through an existing, placed, and/or indwelling catheter 24 (i.e., when the fluid transfer device 14 is coupled thereto) such that at least an end portion of the flexible tube 74 is disposed in a distal position relative to the indwelling catheter 24. Moreover, with indwelling catheters each having a shape, size, and/or configuration that can vary based on, for example, a manufacturer of the indwelling catheter 24 and/or its intended usage, the fluid transfer device 14 can be arranged to be coupled to an indwelling catheter 24 having any suitable configuration and subsequently, to advance at least a portion of a tube through the indwelling catheter 24. In addition, the fluid transfer device 14 can be manipulated by a user to place a distal surface of the flexible tube 74 a predetermined and/or desired distance beyond a distal surface of the indwelling catheter 24 to be disposed within a portion of a vein that is targeted to receive an administered fluid.

The housing 70 of fluid transfer device 14 can be an elongate member having a proximal end 80 and a distal end 82 and defining an inner volume 83. In some embodiments, the housing 70 may be formed of a pair of housing portions 70a, 70b that are coupled together to define the inner volume 83. The housing 70 may include one or more features or surface finishes on an outer surface thereof that can be arranged to increase the ergonomics of the fluid transfer device 14, which in some instances can allow a user to manipulate the fluid transfer device 14 with one hand (i.e., single-handed use). The distal end 82 of the housing 70 can include a coupler 84 formed thereon configured to mate with the coupling device 72 of fluid transfer device and, in one embodiment, the coupler 84 may include a threaded inner surface that engages the coupling device 72. Additionally, the proximal end 80 of the housing 70 can include an opening or port 85 configured to receive a portion of the secondary catheter 76 (i.e., the portion of the secondary catheter 76 extending proximally out from housing 70) and provide for advancement and retraction of the secondary catheter 76 through the port 85.

The coupling device 72 of fluid transfer device 14 is provided at the distal end 82 of the housing 70, with the coupling device 72 providing for reversible coupling of the fluid transfer device 14 to catheter assembly 12, such as via proximal coupler 30 as shown in FIG. 1. As previously described, proximal coupler 30 may be provided as a needleless access connector and is thus referred to hereafter as “needleless access connector 30.” In some embodiments, the coupling device 72 is configured as a lock 86 that includes a blunted cannula 88 and locking arms 90 for coupling to the needleless access connector 30 of catheter assembly 12, with the blunted cannula 88 and locking arms 90 forming three points of contact therewith. However, those of skill will appreciate that any connection or coupling, for example a luer, can be used, so long as a distal end of flexible tube 74 may pass through the coupling device 72 to access catheter assembly 12. Additionally, a proximal end of the coupling device 72 may be configured as a threaded connection 91 (i.e., a threaded outer surface) that is configured to engage the coupler 84 of housing 70—with the threaded connection 91 of coupling device 72 engaging the threaded inner surface of coupler 84.

In accordance with some aspects of the disclosure, the advancement member 78 of fluid transfer device 14 includes a first portion 92 and a second portion 94. The first portion 92 is movably disposed along an upper surface 96 of the housing 70 and the second portion 94 is movably disposed within the inner volume 83 of the housing 70. The arrangement of the advancement member 78 and the housing 70 is such that a connecting portion (not shown) of the advancement member 78 that joins the first and second portions 92, 94 is seated within a slot 98 formed in the upper surface 96 of the housing 70—the slot 98 generally extending between the proximal and distal ends 80, 82 of the housing 70. As the first and second portions 92, 94 are joined together, movement of the first portion 92 along the upper surface 96 of the housing 70 results in a corresponding movement of the second portion 94 within the inner volume 83.

As shown in FIGS. 2 and 3, the first portion 92 of the advancement member 78 may be configured as a tab 100 having a contact surface engageable by a user and an underside that is in contact with the outer surface 96 of the housing 70. In such embodiments, the upper surface 96 of the housing 70 can include a track 101, for example, a set of ribs, ridges, bumps, grooves, and/or the like along which the underside of tab or protrusion advances when the advancement member 78 is engaged by a user. In this manner, a user can engage the first portion 92 of the advancement member 78 and can move the advancement member 78 relative to the housing 70.

As further shown in FIG. 3, the second portion 94 includes an opening 102 extending therethrough that is configured to grip or retain a portion of the flexible tube 74. Due to a portion of the flexible tube 74 being retained within the opening 102 of second portion 94, movement of the advancement member 78 relative to housing 70 causes a corresponding movement of the flexible tube 74 relative to the housing 70. In this manner, a distal end 104 of the flexible tube 74 can be selectively moved out of or back into the inner volume 83 of the housing 70 as desired, such as advancing the distal end 104 of the flexible tube 74 out of the housing 70 when the fluid transfer device 14 has been coupled to the catheter assembly 12 and a fluid infusion/delivery procedure is to be performed.

In addition to receiving flexible tube 74 therein, the second portion 94 also grips or retains a portion of secondary catheter 76 therein. That is a distal end 106 of secondary catheter 76 is secured to second portion 94 (i.e., in opening 102 thereof) and extends proximally out therefrom, with the secondary catheter 76 extending out through opening 85 of the housing 70. A proximal end 108 of the secondary catheter 76 may include a coupler 110 thereon that may be coupled to a (first) fluid source 112 (FIG. 1), such as a fluid reservoir or pre-filled syringe, with the secondary catheter 76 establishing fluid communication between the fluid source 112 and the flexible tube 74. Additionally, in some embodiments, the secondary catheter 76 may also have a clamp 114 provided thereon, such as a slide clamp or pinch clamp, to facilitate stoppage of flow through the secondary catheter 76.

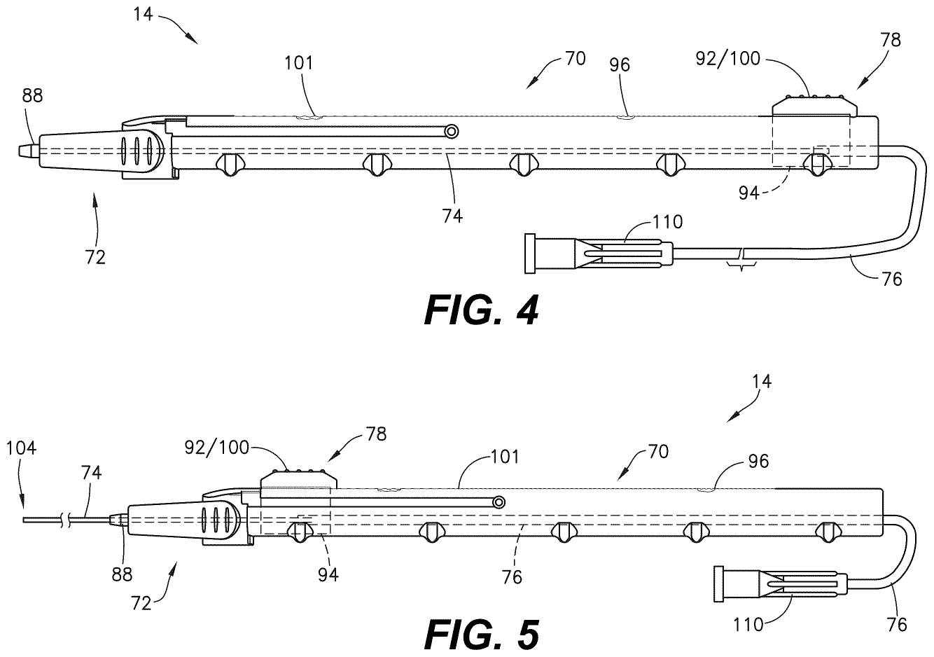

Referring now to FIGS. 4 and 5, changing of the fluid transfer device 14 between the first configuration and second configuration is shown. The fluid transfer device 14 can be in the first configuration prior to use and can be transitioned by a user (e.g., a doctor, physician, nurse, technician, phlebotomist, and/or the like) from the first configuration (FIG. 4) to the second configuration (FIG. 5) to dispose at least the distal end 104 of the flexible tube 74 in a distal position relative to the housing 70.

The fluid transfer device 14 is in the first configuration of FIG. 4 when the flexible tube 74 is disposed in the first position within the housing 70. In some embodiments, substantially the entire flexible tube 74 is disposed within the housing 70 when the flexible tube 74 is in the first position. In other embodiments, the flexible tube 74 is disposed within the housing 70 and the lock 86 when flexible tube 74 is in the first position.

The advancement member 78 is disposed in a proximal position when the fluid transfer device 14 is in the first configuration, and the user may engage the tab 100 of advancement member 78 to move the advancement member 78 relative to the housing 70, which in turn, moves the flexible tube 74 from the first position (e.g., disposed within the housing 70) toward the second position. In this manner, the flexible tube 74 is moved through the inner volume 83 of housing 70 and through the lock 86 and, as such, at least the distal end 104 of the flexible tube 74 is disposed outside of and distal to the lock 86.

The fluid transfer device 14 is in the second configuration of FIG. 5 when the flexible tube 74 is disposed in the second position. The second position of the flexible tube 74 is reached when the distal end 104 of the flexible tube 74 is placed in a desired position relative to a distal tip of the catheter 24. In some instances, for example, a distal end 104 of the flexible tube 74 can be substantially flush with a distal tip of the catheter 24 when the flexible tube 74 is in the second position. In other instances, the distal end 104 of the flexible tube 74 can extend a predetermined distance beyond the distal tip of the catheter 24, such that the distal end 104 of the flexible tube 74 is positioned within the vein at a predetermined distance beyond the distal end of the flexible tube 74.

With the flexible tube 74 in the second position (e.g., with the fluid transfer device 14 in the second configuration shown, for example, in FIG. 5), the user can establish fluid communication between the fluid source 112 and the flexible tube 74. The fluid transfer device 14 can then deliver/infuse a volume of fluid from fluid source 112 into the vein of the patient based at least in part on disposing the distal end 104 of the flexible tube 74 at the predetermined and/or desired distance beyond the distal surface of the catheter 24 (FIG. 1). In some instances, once a desired volume of a drug has been delivered to the patient, the user can move the advancement member 78 in the proximal direction, such as moving the advancement member 78 back to its proximal most position, so as to move the flexible tube 74 back to the first position.

While fluid transfer device 14 is shown and described in FIGS. 1-5 as having an advancement member 68 having first and second portions 92, 94 configured formed to move along an upper surface 96 of housing 70 and within the inner volume 83 thereof, it is recognized that fluid transfer device 14 may have an advancement member of alternate configurations. For example, fluid transfer device 14 may be configured to have a nested housing arrangement-where the advancement member is provided as a telescopic cylinder that may move within housing 70 to advance (and retract) flexible tube 74, according to another embodiment. Examples of fluid transfer devices that include alternate advancement member configurations are provided in US Patent Publication Nos. US2024/0075253 and US2024/0075249, assigned to Becton, Dickinson and Company, which are incorporated herein by reference in their entirety. It is recognized that still other advancement member configurations may be implemented with fluid transfer device 14.

According to aspects of the disclosure, one potential use of the system 10 is for the infusion or administering of a caustic medication, such as a chemotherapy drug or antibiotic. When infusing a caustic medication, it is known that infusion of such a caustic medication may damage the tunica intima layer of a vein, especially when delivery of such medication is to smaller diameter peripheral veins. Accordingly, fluid transfer device 14 may be specifically configured to provide for infusion of a caustic medication, while limiting/reducing the harmful effects of caustic medications on vein walls.

According to one aspect of the disclosure, the flexible tube 74 is specifically constructed to provide for dilution of the caustic medication, so as to protect the vasculature adjacent the distal tip 116 of the catheter 24 from the caustic medication. In particular, a sizing/thickness of the flexible tube 74 is controlled relative to a sizing of the catheter 24, to ensure that a sheath flow of non-caustic medication or saline can be administered simultaneously with administration of the caustic medication, such that the caustic medication can be diluted by a desired amount.

According to embodiments of the disclosure, the flexible tube 74 may be composed of a material that allows for manufacturing of the flexible tube 74 with tight tolerances and with features therein that allow for control of a desired fluid flow through the flexible tube 74 and/or catheter 24. Non-limiting examples of materials from which the flexible tube 74 may be formed include polyamide, polyurethane, TPE, nylon, or other suitable polymeric, elastomeric, or metallic materials that can form a tubing structure.

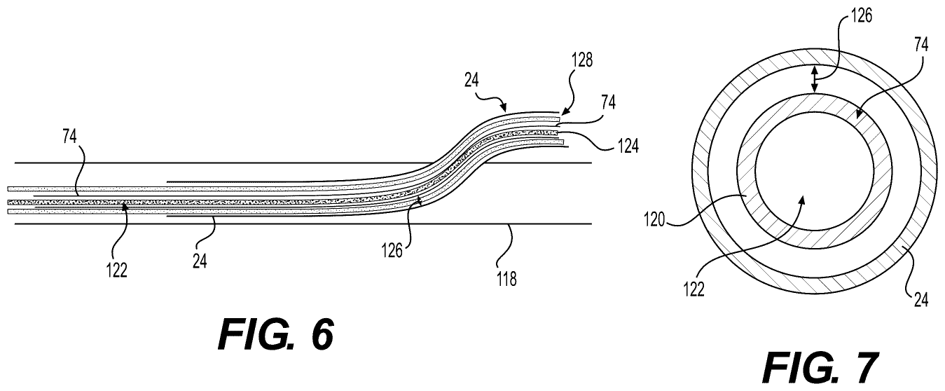

Referring to FIGS. 6 and 7, cross-sectional views of flexible tube 74 and catheter 24 are provided, in isolation and when positioned with the vein 118 of a patient. According to aspects of the disclosure, a thickness of an outer cylindrical wall 120 of the flexible tube 74 is reduced, so as to enable a down-gauging of the flexible tube 74 while still maintaining a desired size of a lumen 122 defined by the flexible tube 74, and thereby still provide for a desired flow rate of caustic medication 124 through the flexible tube 74. With the thickness of the tube wall 120 and a gauge of the flexible tube 74 relative to the gauge of catheter 24 controlled/selected in this manner, the size of a gap 126 formed between an outer surface of the flexible tube 74 and an inner surface of the catheter 24 may be controlled/selected—with a gap 126 being formed that allows for a sheath flow of non-caustic medication or saline 128 through the catheter 24 during delivery of a caustic medication 124 through the flexible tube 74. That is, with a gap 126 of sufficient size provided between an outer surface of the flexible tube 74 and an inner surface of the catheter 24, a fluid flow of non-caustic medication or saline 128 may be delivered from fluid source 38, through extension tubing 22, catheter assembly 12, and catheter 24 at the same time as caustic medication 124 is being delivered from fluid source 112 through flexible tube 74 of fluid transfer device 14. This simultaneous infusion of non-caustic medication or saline 128 with the caustic medication 124 provides for dilution of the caustic medication as it is being administered intravenously, which provides protection for the vasculature from the caustic medication 124 at the distal tip of the catheter 24.

According to aspects of the disclosure, the flexible tube 74 may be constructed such that the cylindrical wall 120 thereof has an outer diameter sized to be less than a minimum section of an inner diameter of the catheter 24. The cylindrical wall 120 of flexible tube 74 should have sufficient wall thickness and stiffness to allow passage through the catheter 24, with it recognized that the thickness of the wall 120 may vary depending on the gauge of the flexible tube 74. With the flexible tube 74 constructed as such, a gap 126 is formed that allows for a sheath flow of non-caustic medication or saline through the catheter 24, while still providing for a desired flow rate of caustic medication through the flexible tube 74. In some embodiments, a size of the gap 126 is between 0.0 inches and half of a catheter tip inner diameter. In other embodiments, a size of the gap is between 0% to 90% of the cross sectional area of an area of: (the catheter tip opening—the flexible tubing outer diameter cross sectional area)/the area of the catheter tip opening. With the gap 126 sized as such, a sheath flow of non-caustic medication or saline is provided for.

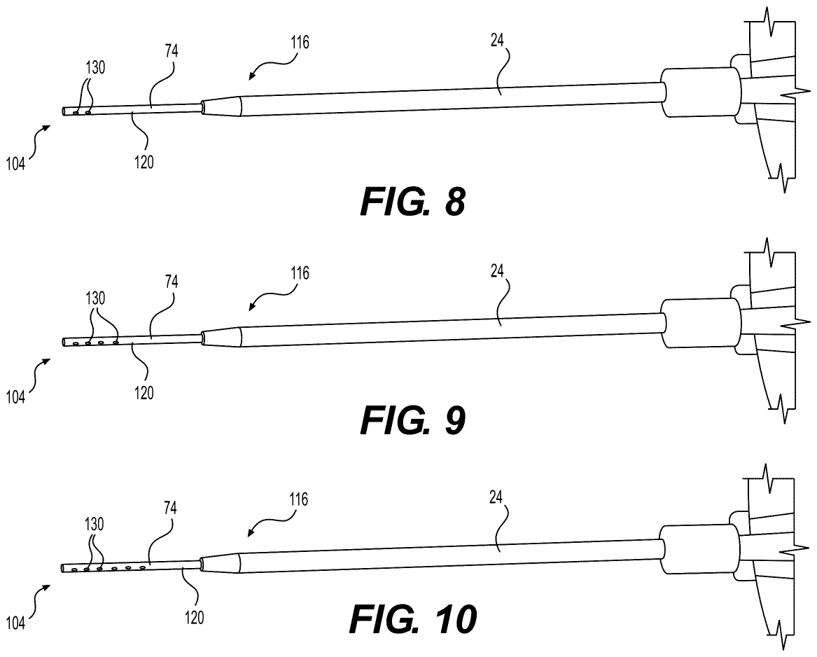

According to another aspect of the disclosure, the flexible tube 74 is specifically constructed to provide for a controlled dispersion of the caustic medication within the vein. In particular, the flexible tube 74 is constructed to include a number of diffuser holes 130 formed in the cylindrical outer wall 120 thereof at or near a distal end 104 of the flexible tube 74—i.e., at the portion of the flexible tube 74 that may be advanced out past the distal tip 116 of the catheter 24. According to embodiments, and as shown in FIGS. 8-10, the diffuser holes 130 may be arranged in any symmetrical or non-symmetrical pattern along a length of the flexible tube 74 near the distal end 104, with it recognized that the number of diffuser holes 130 can be selected based on caustic medication being administered and/or and delivery method of the caustic medication. In general, the number of diffuser holes 130 formed in the flexible tube 74 may be higher when it is desired to more greatly disperse delivery of the caustic medication, while the number of diffuser holes 130 formed in the flexible tube 74 may be lower when it is desired to target delivery of the caustic medication to a more targeted location.

In the embodiment of FIG. 8, a series of two (2) diffuser holes 130 is arranged along a length of the flexible tube 74, near the distal end 104. The series of two diffuser holes 130 may provide a very targeted delivery of the caustic medication within the vein.

In the embodiment of FIG. 9, a series of six (6) diffuser holes 130 is arranged along a length of the flexible tube 74, near the distal end 104. The series of six diffuser holes 130 may provide more dispersed delivery of the caustic medication along a greater length within the vein.

In the embodiment of FIG. 10, a series of four (4) diffuser holes 130 is arranged along a length of the flexible tube 74, near the distal end 104. The series of four diffuser holes 130 may provide a mixture of dispersed and targeted delivery of the caustic medication within the vein.

According to embodiments, it is recognized that the feature of the diffuser holes 130 formed in the flexible tube 74 at or near the distal end 104 thereof may be combined with the feature of the flexible tube 74 having a reduced thickness (i.e., reduced thickness of the outer wall 120). According to embodiments of the disclosure, the diffuser holes 130 may be distributed along the length and/or circumference of the flexible tubing 74. The diffuser holes 130 may be cut perpendicularly through the tubing wall or may be angled distally or proximately. The shape of diffuser holes 130 may be circular, oval, teardrop, or other suitable shape for administering of the caustic medication. Accordingly, based on the selection of any/all or the described variables, the administering of the caustic medication may be controlled as desired.

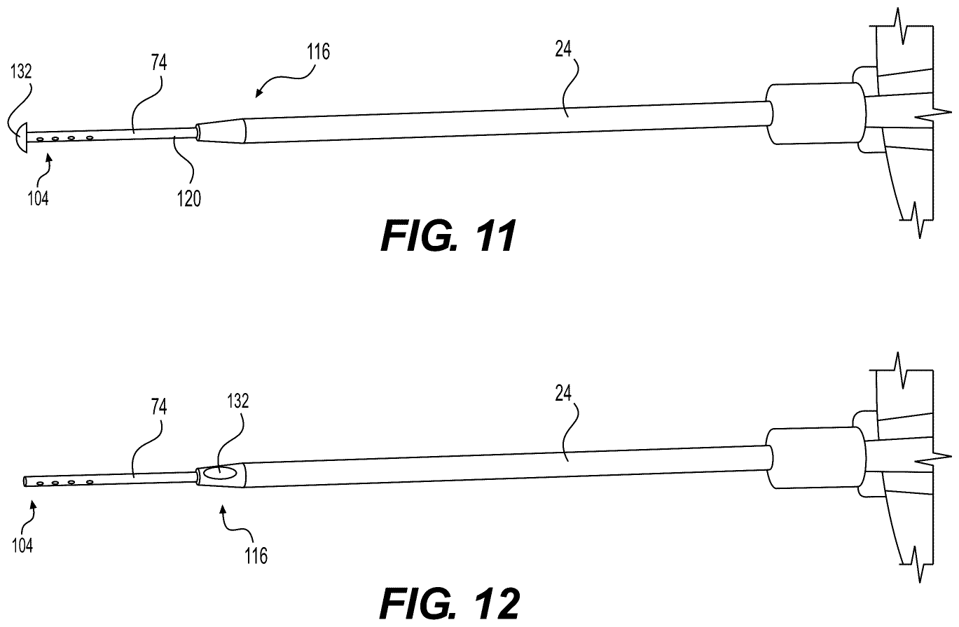

According to another aspect of the disclosure, the flexible tube 74 is configured to enable monitoring of a concentration of the caustic medication at different positions along a length of the flexible tube 74 or near the tip of the catheter 24, to allow clinicians to closely monitor (and adjust positioning or dosage of) the caustic medication. As shown in FIGS. 11 and 12, the flexible tube 74 and/or the catheter 24 may include a sensor 132 thereon configured to measure and monitor a parameter of the caustic medication. In one embodiment, and as shown in FIG. 11, the sensor 132 is provided on the flexible tube 74, such as at the distal end 104 thereof or at another location along a length of the flexible tube 74. In another embodiment, and as shown in FIG. 12, the sensor 132 is provided on the catheter 24, at or near the distal tip 116 thereof.

According to embodiments, the sensor 132 may comprise a pH sensor or osmolarity sensor, as non-limiting examples, that measures and provides feedback to a clinician on the concentration (pH or osmolarity) of the caustic medication at different positions within the vein and may be configured to wirelessly transmit measurements of such parameters to a remote receiver. The sensor 132 may be configured to take measurements of the pH and/or osmolarity at regular intervals, so as to enable a clinician to closely monitor and adjust the positioning and/or dosage of the caustic medication administered to the patient as needed, in order to prevent the caustic medication damaging the vein walls.

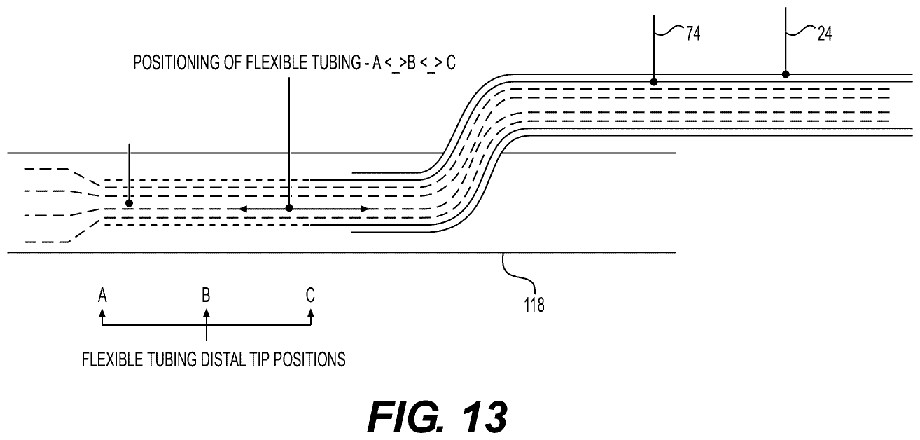

According to another aspect of the disclosure, the fluid transfer device 14 may be controlled during operation, either manually or semi-automatically (e.g., using an externally-controlled motorized mechanism), to vary/adjust the positioning of the distal end 104 of flexible tube 74 within the vein 118 during infusion of the caustic medication, in order to optimize delivery of the caustic medication and maintain vein health. That is by actuating advancement member 78 of fluid transfer device 14, the flexible tube 74 may be pulled/pushed relative to the catheter 24 (and within the vein) throughout the duration of a caustic medication infusion/therapy, such that the distal end 104 of the flexible tube 74 may be displaced/shifted within the vein and the administering of the caustic medication may be de-localized, which lessens the risk of tissue damage in the tunica intima layer. For example, and as shown in FIG. 13, the location of the distal end 104 of the flexible tube 74 may be shifted between a Position A, Position B, and Position C during administering of the caustic medication via actuation of the fluid transfer device 14, to de-localize delivery of the caustic medication and maintain vein health. Hence, faster healing of the vein (i.e., of the tunica intima) may be attained compared to infusion of the caustic medication at only a single location within the vein.

With the system 10 configured as described above, a method for operating the system 10 to provide for administering of a caustic medication to a patient may be described as follows.

In a first step of operation of system 10, the fluid transfer device 14 may be prepared (e.g., removed from packaging) and the fluid transfer device 14 may be attached to the catheter assembly 12, such as via connection of coupling device 72 of fluid transfer device 14 to proximal coupler 30 of catheter assembly 12.

With the fluid transfer device 14 attached to the catheter assembly 12, a first fluid source 112 containing a caustic medication may be attached to the fluid transfer device 14. In some embodiments, the first fluid source 112 containing the caustic medication may be provided as a pre-filled syringe, with the syringe coupled with the proximal coupler 110 provided on the secondary catheter 76 of fluid transfer device 14. The first fluid source 112 may be in fluid communication with the flexible tube 74, to enable a fluid infusion of the caustic medication to the patient.

In some embodiments, upon attaching of the fluid transfer device 14 to the catheter assembly 12, a second fluid source 38 containing a non-caustic medication or saline solution may be attached to the catheter assembly 12. In some embodiments, the second fluid source 38 containing the non-caustic medication or saline solution may be provided as a pre-filled syringe, with the syringe coupled with the proximal access port 36 of extension set 18. The second fluid source 38 may be in fluid communication with the catheter 24, to enable a fluid infusion of the non-caustic medication or saline solution to the patient.

After connection of the first fluid source 112 (and, optionally, the second fluid source 38) to the fluid transfer device 14 (and catheter assembly 12), the flexible tube 74 may be advanced into/through the indwelling catheter 24 of catheter assembly 12, as described in detail above. Upon the distal end 104 of the flexible tube 74 being positioned out past the distal tip 116 of the catheter 24 and at a desired location within the vein, the caustic medication may then be administered to the patient via actuation/operation of the first fluid source 112. In some embodiments, the non-caustic medication or saline solution may be simultaneously administered to the patient along with the caustic medication via actuation/operation of the second fluid source 38, in order to dilute the caustic medication and limit potential damage to the vein wall.

According to aspects of the disclosure, the structure of the flexible tube 74 in fluid transfer device 14 and/or the operation of the fluid transfer device 14 provide for infusion of the caustic medication while limiting/reducing the harmful effects of the caustic medication on the vein walls. As previously described above, in one embodiment, a wall thickness and/or gauging of the flexible tube 74 may be selectively controlled/provided (relative to the catheter sizing), to enable a sheath flow of saline solution simultaneously with the fluid flow of the caustic medication, in order to minimize potential damage to the vein from the caustic medication. In another embodiment, the flexible tube 74 may include a number of diffuser holes 130 formed in the cylindrical outer wall 120 of the flexible tube 74 at or near a distal end 104 thereof, in order to provide for a controlled dispersion of the caustic medication within the vein, and in order to minimize potential damage to the vein from the caustic medication. In another embodiment, the flexible tube 74 and/or catheter 24 may include a sensor 132 thereon at one of a number of positions along a length of the flexible tube 74 or near the distal tip 116 of the catheter 24, to allow clinicians to closely monitor pH and/or osmolarity of the caustic medication, and to enable a clinician to adjust the location of the administering of the caustic medication as necessary and/or adjust the dosage of the caustic medication.

Beneficially, embodiments of the disclosure provide a system and method for infusion of caustic medications into an indwelling PIVC, while reducing the harmful effects of caustic medications on the vein wall. The fluid transfer device enables variation in the location of intravenous administration of the caustic medication, with such distributed delivery lessening the risk of tissue damage to the vein. The fluid transfer device also enables dilution of the caustic medication upon infusion, to again lessen the risk of tissue damage to the vein at the point of infusion. The fluid transfer device still further allows for monitoring of a concentration of the caustic medication at different positions along the length of the flexible tube or near the tip of the PIVC, to allow clinicians to closely monitor and adjust positioning or dosage of the caustic medication.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A system for intravenous delivery of a caustic medication to a patient, the system comprising:
a catheter assembly including a catheter adapter and a catheter positionable intravenously in the patient;
a fluid source containing a caustic medication in fluid form; and
a fluid transfer device coupleable to the fluid source and to the catheter assembly, the fluid transfer device comprising:
a flexible tube configured to be inserted within the catheter, the flexible tube in fluid communication with the fluid source;
a housing having a proximal end and a distal end and defining an inner volume configured to movably receive the flexible tube;
a coupling device positioned at the distal end of the housing and configured to couple the housing to the catheter assembly; and
an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer fluid from the fluid source to the patient when in the second position;
wherein the fluid transfer device is configured to control one or more of an intravenous location or dispersion of the caustic medication infused to the patient, and a dilution of the caustic medication infused to the patient.

2. The system of claim 1, wherein the flexible tube comprises a cylindrical outer wall, with the cylindrical outer wall having an outer diameter sized to be less than a minimum section of the catheter inner diameter, with sufficient wall thickness and stiffness to allow passage through the catheter.

3. The system of claim 2, wherein a gauge of the catheter is a gauge of the catheter is between 14 G and 26 G and a gauge of the flexible tube is less than the gauge of the catheter, such that a gap is provided between an outer surface of the flexible tube and an inner surface of the catheter.

4. The system of claim 3, wherein a width of the gap is between 0.0 inches and half of a catheter tip inner diameter.

5. The system of claim 3, wherein the catheter adapter comprises a proximal port and a side port, with the fluid transfer device connected to one of the proximal port or a side port via the coupling device.

6. The system of claim 5, further comprising a second fluid source coupled to the other of the proximal port or the side port and in fluid communication with the catheter, the second fluid source containing a non-caustic medication or saline solution.

7. The system of claim 6, wherein the non-caustic medication or saline solution is infused through the catheter to the patient via a sheath flow through the gap between the outer surface of the flexible tube and the inner surface of the catheter.

8. The system of claim 1, wherein the flexible tube comprises a plurality of diffuser holes formed therein at the distal end of the flexible tube.

9. The system of claim 8, wherein the plurality of diffuser is distributed along a length and/or circumference of the flexible tubing.

10. The system of claim 1, further comprising a sensor positioned at the distal end of the flexible tube or the distal tip of the catheter configured to measure a concentration of the caustic medication infused to the patient.

11. The system of claim 10, wherein the sensor comprises a pH sensor.

12. The system of claim 10, wherein the sensor comprises an osmolarity sensor.

13. The system of claim 1, wherein the fluid transfer device comprises a secondary catheter extending proximally out from the housing, the secondary catheter including a connector at a proximal end thereof, with the fluid source coupled to the connector on the secondary catheter.

14. A method of intravenously delivering a caustic medication to a patient, the method comprising:
connecting a fluid transfer device to a catheter assembly including a catheter adapter and a catheter having a distal tip that is indwelling intravenously in the patient, the fluid transfer device comprising:
a flexible tube configured to be inserted within the catheter, the flexible tube in fluid communication with the fluid source;
a housing having a proximal end and a distal end and defining an inner volume configured to movably receive the flexible tube;
a coupling device positioned at the distal end of the housing and configured to couple the housing to the catheter assembly; and
an advancement member configured to move relative to the housing to move the flexible tube between a first position, in which a distal end of the flexible tube is disposed within the housing or within the coupling device, and a second position, in which the distal end of the flexible tube is disposed within the catheter or past a distal tip of the catheter, with the flexible tube able to transfer fluid from the fluid source to the patient when in the second position;
connecting a fluid source to the fluid transfer device such that the fluid source is fluidly connected with the flexible tube, wherein the fluid source contains a caustic medication in fluid form;
actuating the advancement member to move the flexible tube from the first position to the second position; and
with the flexible tube in the second position, operating the fluid source to administer the caustic medication to the patient, via the flexible tube;
wherein, in administering the caustic medication to the patient, the fluid transfer device controls one or more of an intravenous location or dispersion of the caustic medication administered to the patient, and a dilution of the caustic medication administered to the patient.

15. The method of claim 14, further comprising:
connecting another fluid source to the catheter assembly via a side port of the catheter adapter and such that the another fluid source is fluidly connected with the catheter, wherein the another fluid source contains a non-caustic medication or saline solution; and
operating the another fluid source to administer the non-caustic medication or saline solution to the patient, via the catheter.

16. The method of claim 15, wherein the non-caustic medication or saline solution is administered to the patient simultaneously with administration of the caustic medication, with the non-caustic medication or saline administered via a sheath flow through a gap between an outer wall of the flexible tube and an inner surface of the catheter.

17. The method of claim 16, wherein a size of the gap is between 0.0 inches and half of a catheter tip inner diameter.

18. The method of claim 14, wherein, in administering the caustic medication to the patient, the caustic medication is infused through diffuser holes formed in an outer wall of the flexible tube, at the distal end of the flexible tube.

19. The method of claim 14, further comprising monitoring a parameter of the caustic medication during administering thereof via a sensor positioned at the distal end of the flexible tube or at the distal tip of the catheter, wherein the parameter comprises one of a pH or osmolarity of the caustic medication.

20. The method of claim 14, further comprising actuating the advancement member during administering of the caustic medication to move the distal end of the flexible tube to a plurality of positions that are all distal to the distal tip of the catheter, to vary an intravenous location within the patient for de-localized delivery of the caustic medication.
